(19) United States
(12) Patent Application Publication
(10) Pub. No.: US 2005/0064488 A1
(43) Pub. Date: Mar. 24, 2005

(54) Method for detecting a target nucleic acid by using a detection probe capable of hybridizing with a tag sequence

(76) Inventors: Nam Huh, Seoul (KR); Sung-young Jeong, Yongin-si (KR); Kui-hyun Kim, Daejeon-si (KR)

Correspondence Address: CANTOR COLBURN, LLP, 55 GRIFFIN ROAD SOUTH, BLOOMFIELD, CT, 06002, US

(21) Appl. No.: 10/945,245

(22) Filed: Sep. 20, 2004

(30) Foreign Application Priority Data
Sep. 19, 2003 (KR) 2003-65218

(51) Int. Cl. C12Q 1/68; C12P 19/34

(52) U.S. Cl. 435/6; 435/91.2

(57) ABSTRACT

Provided is a method for detecting a target nucleic acid, comprising amplifying a target nucleic acid from a nucleic acid sample using a set of primers comprising at least one primer having a tag sequence at its 5′ end and a target binding sequence at its 3′ end; hybridizing the resulting amplified products with detection probes labeled with a label, the detection probes being capable of specifically binding to the tag sequence; hybridizing the hybridization products with capture probes immobilized on a microarray, the capture probes being capable of specifically binding to the target nucleic acid and not to the tag sequence; and determining the results of hybridization.

### BACKGROUND OF THE INVENTION

This application claims priority from Korean Patent Application No. 2003-65218 filed on Sep. 19, 2003, in the Korean Intellectual Property Office, the disclosure of which is incorporated herein in its entirety by reference.

1. Field of the Invention

The present invention relates to a method for detecting target nucleic acid in an efficient manner.

2. Description of the Related Art

Methods for amplifying a nucleic acid are potent techniques for rapidly detecting a specific target sequence and are known in the art. Examples of such methods include polymerase chain reaction (PCR) [U.S. Pat. Nos. 4,683,195; 4,683,202; 4,800,159 and 4,965,188], strand displacement amplification (SDA) [U.S. Pat. No. 5,270,184], nucleic acid sequence based amplification (NASBA) [U.S. Pat. No. 5,130,238], transcription based amplification [D. Kwoh et al., 1989. Proc. Nat. Acad. Sci. USA 86, 1173-1177], self-sustained sequence replication (3SR) [J. Guatelli et al., 1990. Proc. Nat. Acad. Sci. USA 87,1874-1878], and Qβ replicase system [P. Lizardi et al., 1988. BioTechnology 6, 1197-1202].

Methods for identifying or quantifying the amplified nucleic acids on a microarray include a direct labeling method, a sandwich analysis method, and the like. Among direct labeling methods, a direct dye incorporation method comprises directly labeling PCR products by performing PCR with a PCR reaction solutions containing a monomer labeled with a label, such as fluorescence, for example, a fluorescent labeled dNTP, hybridizing the labeled PCR products to capture probes on a microarray and then reading signals generated from the labels to detect or quantify the target nucleic acid. This method has an advantage that labeling can be performed through PCR without a need for a separate labeling reaction. However, this method has the following drawbacks: labeling efficiency is low and it is difficult to obtain a quantitative relationship between the PCR products obtained by PCR and the signals generated from the PCR products. Further, amplification of the target nucleic acid by a multiplex PCR increases the amount of dyes used, thereby increasing costs. There is another direct labeling method in which the ends of amplification product are labeled with dyes using enzymes such as terminal transferases. However, this method needs a separate enzyme reaction and has a difficulty in maintaining a consistent activity of enzyme, thus resulting in a low reproducibility. An analysis method using stacking is described by C. Mirkin in U.S. Pat. No. 6,582,921, wherein capture probes immobilized on a microarray and labeled detection probes, which specifically bind to target nucleic acids, are used. Specifically, the method comprises amplifying target nucleic acids and hybridizing the amplified target nucleic acids with capture probes immobilized on a microarray and detection probes. The hybridization process is illustrated in FIG.1. Referring to FIG. 1, a target nucleic acid 9 is hybridized with a capture probe 7 immobilized on a microarray 6 and a detection probe 8. Then, the degree of hybridization of the detection probe 8 with target nucleic acid 9 is determined by measuring the signals from the detection probe 8. From the result, the presence or absence of the target nucleic acid and the amount of target nucleic acids can be determined. However, this method requires a detection probe complementary to a specific sequence in the target nucleic acid. Thus, the detection probe can be applied only to the target nucleic acid having a specific sequence, and a mutation in the specific sequence may result in failure. In addition, the method comprising binding a molecule having a high affinity to a specific molecule, such as biotin, to an end of a primer and generating signals, such as fluorescent signals, using the specific molecule, such as fluorescent labeled streptavidine and the like, is known [Affymetrix, U.S. Pat. No. 6,203,989]. However, the method has complicated processes and needs a high level of skilled technique to attain reproducibility in the reaction.

The inventors studied and developed a method for detection of target nucleic acid which can be performed in a simple process without separately bonding labels, such as dyes, to the targets during the amplification process, even when a plurality of target nucleic acids are amplified as in a multiplex PCR.

### SUMMARY OF THE INVENTION

The present invention provides a method for detecting target nucleic acids in a simple and efficient manner using a detection probe which can be universally used for at least one target nucleic acid.

According to an aspect of the present invention, there is provided a method for detecting a target nucleic acid, comprising:
amplifying a target nucleic acid from a nucleic acid sample using a set of primers comprising at least one primer having a tag sequence at its 5′ end and a target binding sequence at its 3′ end;
hybridizing the resulting amplified products with detection probes labeled with a label, the detection probes being capable of specifically binding to the tag sequence;
hybridizing the hybridization products with capture probes immobilized on a microarray, the capture probes being capable of specifically binding to the target nucleic acid and not to the tag sequence; and
determining the results of hybridization.

According to another aspect of the present invention, there is provided a method for detecting a target nucleic acid, comprising:
amplifying a target nucleic acid from a nucleic acid sample using a set of primers comprising at least one primer having a tag sequence at its 5′ end and a target binding sequence at its 3′ end;
hybridizing the resulting amplified products with capture probes immobilized on a microarray, the capture probes being capable of specifically binding to the target nucleic acid and not to the tag sequence;
hybridizing the hybridization products with detection probes labeled with a label, the detection probes being capable of specifically binding to the tag sequence; and
determining the results of hybridization.

According to still another aspect of the present invention, there is provided a method for detecting a target nucleic acid, comprising:
amplifying a target nucleic acid from a nucleic acid sample using a set of primers comprising at least one primer having a tag sequence at its 5′ end and a target binding sequence at its 3′ end;
hybridizing the resulting amplified products with detection probes labeled with a label and capture probes immobilized on a microarray, the detection probes being capable of specifically binding to the tag sequence and the capture probes being capable of specifically binding to the target nucleic acid and not to the tag sequence; and
determining the results of hybridization.

### BRIEF DESCRIPTION OF THE DRAWINGS

The above and other features and advantages of the present invention will become more apparent by describing in detail exemplary embodiments thereof with reference to the attached drawings in which:

FIG. 1 is a view illustrating hybridization of a target nucleic acid with a capture probe and a detection probe, according to a conventional sandwich analysis method;

FIG. 2 is a view illustrating an amplification process of a target nucleic acid using an amplification primer containing a tag, according to a method of the present invention;

FIG. 3 is a view illustrating hybridization of a target nucleic acid with a capture probe and a detection probe, according to a method for detecting a target nucleic acid of the present invention;

FIGS. 4A and 4B respectively show an array of capture probes in a microarray used for the detection of target nucleic acids directly labeled by PCR and a result of the detection of target nucleic acids on the array;

FIG. 4C shows a result of the detection of target nucleic acids according to the method of the present invention; and

FIG. 5 is a view illustrating a result of fluorescent signal analysis of MO1E10-01mp and MO1E10-01wp spots for detecting a mutant type I454Vofexon 10 and a normal type of exon 10.

### DETAILED DESCRIPTION OF THE INVENTION

The term “target nucleic acid”, as used herein, refers to a nucleic acid sequence to be amplified. The term also refers to a nucleic acid sequence comprising a sequence region which may bind to a detection probe attached to a microarray. The term “tag sequence”, as used herein, refers to any sequence which is included in an amplification primer at a 5′ end, which after amplification is to be included in an amplified target nucleic acid; The term “detection probe”, as used herein, refers to a probe which may be hybridized with a specific sequence of a target nucleic acid so as to detect the target nucleic acid. With regard to the invention, the term means a nucleic acid having a sequence which may be hybridized with the tag sequence in the amplified target nucleic acid and when hybridized with the tag sequence, it is possible to detect the presence or absence of the target nucleic acids and the quantitative amount of the target nucleic acids. The detection probe may be labeled with a label. The term “primer”, as used herein, refers to a nucleic acid which may be hybridized to a target nucleic acid in a nucleic acid amplification reaction and act as a point of initiation of elongation reaction of a nucleic acid. The primer used in the present invention includes a tag sequence at its 5′ end and a sequence hybridized to the target nucleic acid in the downstream of the tag sequence. Among a set of primers used in the amplification of a nucleic acid in the present invention, one primer may include the tag sequence and another primer may not. Both forward and reverse primers may also include the tag sequence. If a PCR of a nucleic acid is performed using a reaction mixture containing a plurality of primer sets, as in the multiplex PCR, at least one of forward and reverse primers in each primer set includes a tag sequence. The term “a multiplex PCR”, as used herein, refers to a PCR method in which amplification is carried out with a PCR reaction mixture containing a plurality of primer sets in one tube.

According to an embodiment of the present invention, there is provided a method for detecting a target nucleic acid, comprising: amplifying a target nucleic acid from a nucleic acid sample using a set of primers comprising at least one primer having a tag sequence at its 5′ end and a target binding sequence at its 3′ end; hybridizing the resulting amplified products with detection probes labeled with a label, the detection probes being capable of specifically binding to the tag sequence; hybridizing the hybridization products with capture probes immobilized on a microarray, the capture probes being capable of specifically binding to the target nucleic acid and not to the tag sequence; and determining the results of hybridization.

In the method of the present invention, amplification may be performed by any amplification process using a primer, such as a PCR or SDA method. Preferably, a PCR or SDA method is used. More preferably, a multiplex PCR method is used. In the method of the present invention, the label may be selected from a fluorescent label, a radioactive, a receptor, and a ligand but is not limited thereto. Any substance can be used as a label so long as it generates signals and does not interfere with hybridization between the detection probe and the tag sequence. In the method of the present invention, the results of hybridization may be determined by a signal generated from the label of the detection probes. The signal may be in the form of light, such as fluorescence or radioactive rays, or an indirect signal derived from enzymatic activity.

According to another embodiment of the present invention, there is provided a method for detecting a target nucleic acid, comprising: amplifying a target nucleic acid from a nucleic acid sample using a set of primers comprising at least one primer having a tag sequence at its 5′ end and a target binding sequence at its 3′ end; hybridizing the resulting amplified products with capture probes immobilized on a microarray, the capture probes being capable of specifically binding to the target nucleic acid and not to the tag sequence; hybridizing the hybridization products with detection probes labeled with a label, the detection probes being capable of specifically binding to the tag sequence; and determining the results of hybridization.

In the method of the present invention, amplification may be performed by any amplification process using a primer, such as a PCR or SDA method. Preferably, a PCR or SDA method is used. More preferably, a multiplex PCR method is used. In the method of the present invention, the label may be selected from a fluorescent label, a radioactive, a receptor, and a ligand but is not limited thereto. Any substance can be used as a label so long as it generates signals and does not interfere hybridization between detection probe and tag sequence. In the method of the present invention, the results of hybridization may be determined by a signal generated from the label of the detection probes. The signal may be in the form of light, such as fluorescence or radioactive rays, or an indirect signal derived from enzymatic activity.

According to still another embodiment of the present invention, there is provided a method for detecting a target nucleic acid, comprising: amplifying a target nucleic acid from a nucleic acid sample using a set of primers comprising at least one primer having a tag sequence at its 5′ end and a target binding sequence at its 3′ end; hybridizing the resulting amplified products with detection probes labeled with a label and capture probes immobilized on a microarray, the detection probes being capable of specifically binding to the tag sequence and the capture probes being capable of specifically binding to the target nucleic acid and not to the tag sequence; and determining the results of hybridization.

In the method of the present method, amplification may be performed by any amplification process using a primer, such as a PCR or SDA method. Preferably, a PCR or SDA method is used. More preferably, a multiplex PCR method is used. In the method of the present invention, the label may be selected from a fluorescent label, a radioactive label, a receptor, and a ligand but is not limited thereto. Any substance can be used as a label so long as it generates signals and does not interfere hybridization between detection probe and tag sequence. In the method of the present invention, the results of hybridization may be determined by a signal generated from the label of the detection probes. The signal may be in the form of light, such as fluorescence or radioactive rays, or an indirect signal derived from enzymatic activity.

The length of the primer, tag sequence, detection probe, and capture probe is not specifically limited and may depend on the hybridization conditions, the objects of detection, and the like. The length may preferably be 10 to 200 nucleotides, more preferably 10 to 100 nucleotides, and most preferably 15 to 50 nucleotides.

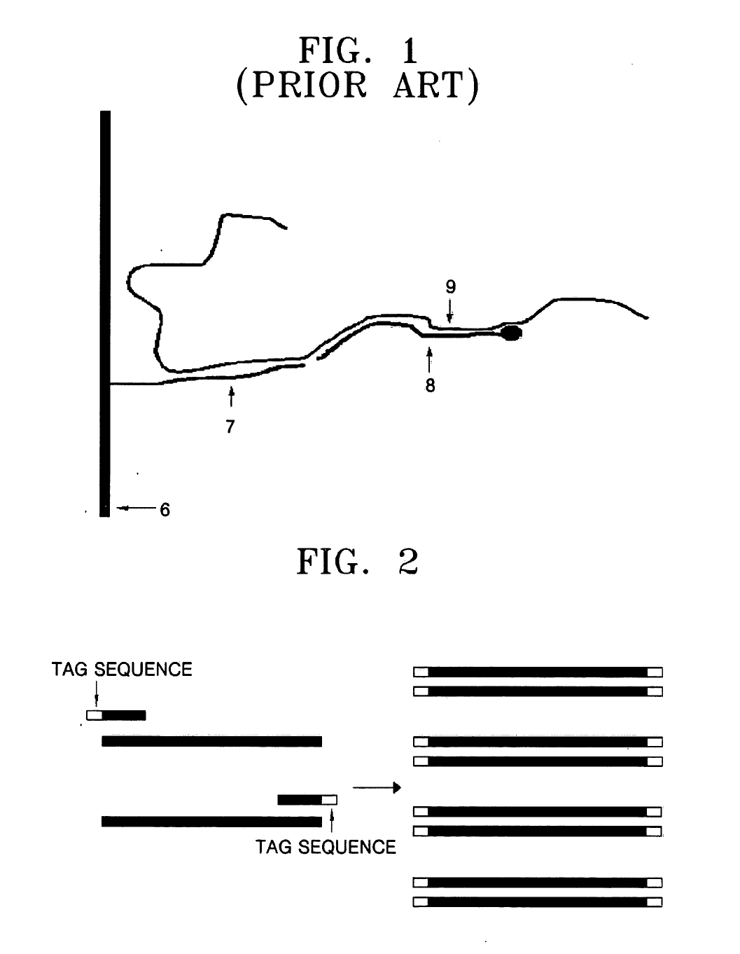

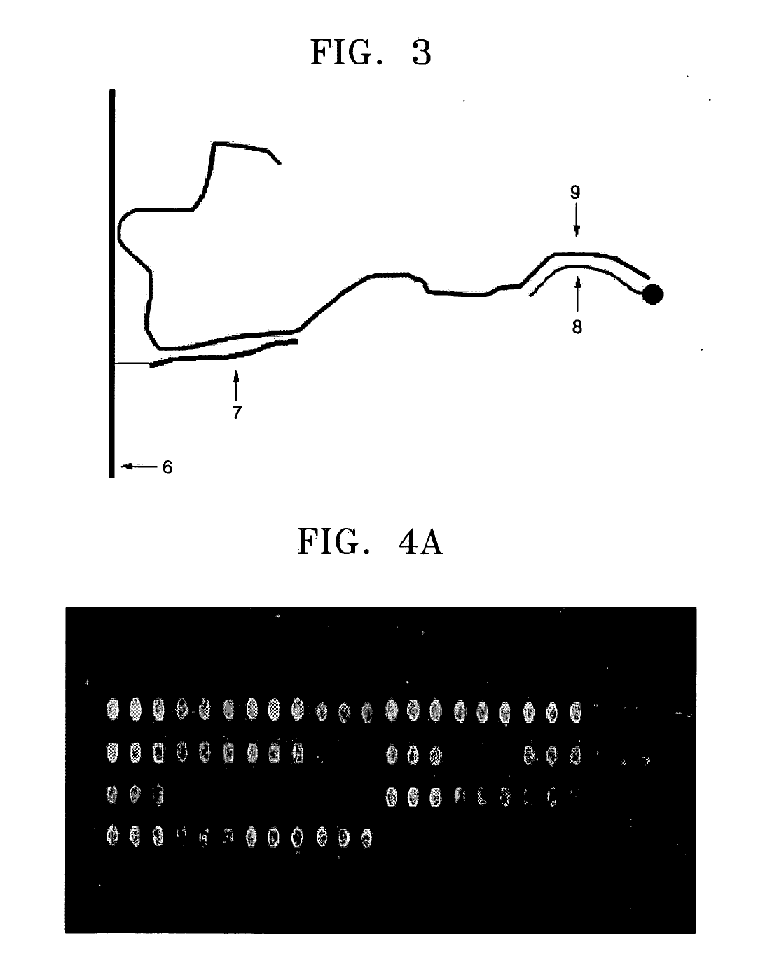

The method of the present invention will be explained with regard to the drawings. FIG. 2 is a view illustrating an amplification process of a target nucleic acid using an amplification primer containing a tag sequence. Through the amplification process, the amplified target nucleic acids comprise the tag sequence(s) at its one end or both ends. FIG. 3 is a view illustrating hybridization of a target nucleic acid 9 with a capture probe 7 immobilized on a microarray 6 and a detection probe 8. As seen from FIG. 3, since the detection probe 8 used in the present invention can bind to the part of tag sequence in the target nucleic acid 9, there is an advantage that the detection probe 8 may be hybridized to those containing the tag sequence irrespective of the sequence information. Thus, the method according to the present invention has a remarkable advantage compared with the conventional analysis method using stacking, which should additionally design and use a detection probe capable of specifically hybridizing to a target nucleic acid and thus is limited in its applications.

Hereinafter, the present invention will be described in more detail with reference to the following example. The example is given for the purpose of illustration and not intended to limit the scope of the invention.

### EXAMPLE

### Example 1

In this example, a target, i.e., MODY 1 gene, was first amplified by PCR using a set of amplification primers containing a tag sequence at the 5′ end. The PCR products were purified and the concentration thereof was measured. The resulting PCR products were hybridized with labeled detection probes which are complementary to the tag sequence. Next, the hybridization products were hybridized with capture probes immobilized on a microarray, the capture probes having a sequence complementary to the target nucleic acid. After the hybridization products were washed, scanning was carried out and then the degree of hybridization was determined to detect the target nucleic acids. The experimental processes of Example 1 are specifically described below.

(1) Amplification of MODY 1 Gene by PCR Using Primers Containing a Tag Sequence

First, a set of primers targeted to seven exons of MODY 1 gene were designed (see, Table 1) (Bioneer, Taejeon, Korea). Each primer of the primer set contains tag sequences 1 and 2 (Sequence ID Nos. 1 and 2) at the 5′ end. The primer set was divided into two groups and subjected to a multiplex PCR using a genome of a normal person as a template. The conditions of the PCR reactions were as follows: the initial denaturation of primers at 94° C. for 3 minutes; the 1^{st} to 7^{th} cycles with one cycle including denaturation at 94° C. for 30 sec, annealing at 64° C. for 15 sec, and extension at 72° C. for 40 sec; the 8^{th} to 29^{th} cycles with one cycle including simultaneous annealing and extension at 72° C. for 3 minutes; and the 30^{th} cycle of final extension at 72° C. for 3 minutes.

TABLE 1
Groups of primer set

| Target and length of products | Primer |
|---|---|
| Exon 2-534 bp | SEQ ID No. 5 |
| | SEQ ID No. 6 |
| Exon 3-324 bp | SEQ ID No. 7 |
| | SEQ ID No. 8 |
| Exon 4-338 bp | SEQ ID No. 9 |
| | SEQ ID No. 10 |
| Exon 7-459 bp | SEQ ID No. 11 |
| | SEQ ID No. 12 |
| Exon 8-506 bp | SEQ ID No. 13 |
| | SEQ ID No. 14 |
| Exon 9-359 bp | SEQ ID No. 15 |
| | SEQ ID No. 16 |
| Exon 10-417 bp | SEQ ID No. 17 |
| | SEQ ID No. 18 |

(2) Hybridization of the PCR Products with Detection Probes and then Hybridization of the Obtained Hybridization Products with the Capture Probes

After the PCR products were purified, the purified PCR products, detection probes 1 (SEQ ID No. 3), and detection probes 2 (SEQ ID No. 4) were mixed with a buffer for hybridization. The detection probes 1 and 2 contained Cy3 dye attached to the 5′ end, as seen in SEQ ID Nos. 3 and 4. The final concentrations of the PCR products and each of the detection probes were adjusted to 100 nM.

Then, the hybridization products of the PCR products and the detection probes were hybridized to capture probes complementary to the PCR products, the capture probes being immobilized on a microarray. The type of mutants observed in MODY1 gene, which may be detected on the microarray, and the capture probes capable of detecting each of the mutants are shown in Table 2. The probes were arranged on the microarray so that they had triple spots, respectively. The hybridization reaction on the microarray was performed in 6×SSPET buffer at 42° C. for 16 hours. After the completion of hybridization reaction, the products were washed with 6×SSPET buffer solution for 5 minutes and 3×SSPET buffer solution for 5 minutes. The hybridization results on the microarray were scanned using an Axon 4000B LASER scanner, and then an imaging process was performed.

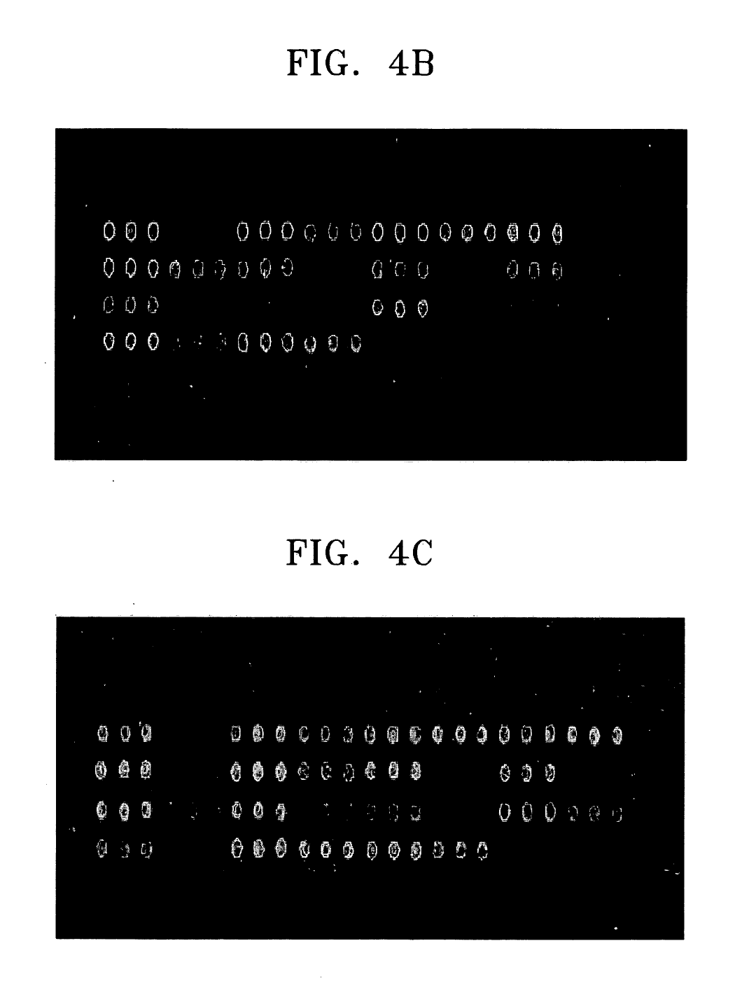

The obtained images are shown in FIG. 4A. The arrangement of the capture on a microarray in FIG. 4A is described in Table 3.

TABLE 2
Mutants of MODY1 gene and capture probes capable of detecting the
mutants of MODY1 gene

| Location | Name of | | |
|---|---|---|---|
| of exon | mutants | Characteristics | Capture probe |
| 2 | D69A | GAC -> GCC | MO1E02-01wp (SEQ ID No. 19: normal type) |
| | | | MO1E02-01mp (SEQ ID No. 20: mutant type) |
| 2 | F75fsdelT | TTC -> TC | MO1E02-02wp (SEQ ID No. 21: normal type) |
| | | | MO1E02-02mp (SEQ ID No. 22: mutant type) |
| 3 | K99delAA | GAC aaG -> GAC G | MO1E03-01wp (SEQ ID No. 23: normal type) |
| | | | MO1E03-01mp (SEQ ID No. 24: mutant type) |
| 3 | G115S | GGC -> AGC | MO1E03-02wp (SEQ ID No. 25: normal type) |
| | | | MO1E03-02mp (SEQ ID No. 26: mutant type) |
| 4 | V121I | GTC -> ATC | MO1E04-01wp (SEQ ID No. 27: normal type) |
| | | | MO1E04-01mp (SEQ ID No. 28: mutant type) |
| 4 | R127W | CGG -> TGG | MO1E04-02wp (SEQ ID No. 29: normal type) |
| | | | MO1E04-02mp (SEQ ID No. 30: mutant type) |
| 4 | R154X | CGA -> TGA | MO1E04-03wp (SEQ ID No. 31: normal type) |
| | | | MO1E04-03mp (SEQ ID No. 32: mutant type) |
| 4 | R154Q | CGA -> CAA | MO1E04-04wp (SEQ ID No. 33: normal type) |
| | | | MO1E04-04mp (SEQ ID No. 34: mutant type) |
| 7 | V255M | GTG -> ATG | MO1E07-01wp (SEQ ID No. 35: normal type) |
| | | | MO1E07-01mp (SEQ ID No. 36: mutant type) |
| 7 | Q268X | CAG -> TAG | MO1E07-02wp (SEQ ID No. 37: normal type) |
| | | | MO1E07-02mp (SEQ ID No. 38: mutant type) |
| 7 | E276Q | GAG -> CAG | MO1E07-03wp (SEQ ID No. 39: normal type) |
| | | | MO1E07-03mp (SEQ ID No. 40: mutant type) |
| 8 | R324H | CGC -> CAC | MO1E08-01wp (SEQ ID No. 41: normal type) |
| | | | MO1E08-01mp (SEQ ID No. 42: mutant type) |
| 9 | V393I | GTC -> ATC | MO1E09-01wp (SEQ ID No. 43: normal type) |
| | | | MO1E09-01mp (SEQ ID No. 44: mutant type) |
| 10 | I454V | ATC -> GTC | MO1E10-01wp (SEQ ID No. 45: normal type) |
| | | | MO1E10-01mp (SEQ ID No. 46: mutant type) |

TABLE 3
Arrangement of capture probes on microarray

| | Column 1 | Column 2 | Column 3 | Column 4 | Column 5 | Column 6 | Column 7 | Column 8 |
|---|---|---|---|---|---|---|---|---|
| Row 1 | MO1E02- | MO1E02- | MO1E02- | MO1E02- | MO1E03- | MO1E03- | MO1E03- | MO1E03- |
| | 01wp | 01mp | 02wp | 02mp | 01wp | 01mp | 02wp | 02mp |
| Row 2 | MO1E04- | MO1E04- | MO1E04- | MO1E04- | MO1E04- | MO1E04- | MO1E04- | MO1E04- |
| | 01wp | 01mp | 02wp | 02mp | 03wp | 03mp | 04wp | 04mp |
| Row 3 | MO1E07- | MO1E07- | MO1E07- | MO1E07- | MO1E07- | MO1E07- | MO1E08- | MO1E08- |
| | 01wp | 01mp | 02wp | 02mp | 03wp | 03mp | 01wp | 01mp |
| Row 4 | MO1E09- | MO1E09- | MO1E10- | MO1E10- | | | | |
| | 01wp | 01mp | 01wp | 01mp | | | | |

(3) Hybridization of the PCR Products with Capture Probes and then Hybridization of the Obtained Hybridization Products with the Detection Probes

The PCR amplified products were first hybridized with the capture probes on the microarray, and then the obtained hybridization products were hybridized with the detection probes and scanned by a scanner.

The hybridization reaction on the microarray was performed in 6×SSPET buffer at 42° C. for 16 hours. After the completion of hybridization reaction, the products were washed with 6×SSPET buffer solution and 3×SSPET buffer solution for 5 minutes each. Next, to the hybridization products, the detection probes dissolved in the hybridization buffer solution (the final concentration of 20 nM) were secondly hybridized at room temperature for one hour. Then, washing was performed as described above. The hybridization results on the microarray were scanned using an Axon 4000B LASER scanner, and then an imaging process was performed.

The results are shown in FIG. 4B. The arrangement of the capture probes on the microarray in FIG. 4B is described in Table 4.

(4) PCR Amplification of the Target Products by a Direct Labeling and Hybridization of the Amplified Products

To compare the detection method of the present invention with a conventional detection method, the target products were amplified through a conventional PCR method by a direct labeling and then hybridized with capture probes on a microarray to detect the amplified products.

First, the composition of a direct labeling reagent used in a direct labeling method was as follows: water (dH₂O) 27.9 μl; 10× buffer, 5 μl; dNTP(dA, dG, dC=200 μM, dT=40 μM), 0.5 μl; template DNA (200 ng/μl), 1 μl; Taq polymerase (3U), 0.6 μl; primer (200 nM), 14 μl; and cy3-dUTP (20 μM), 1 μl. The conditions of the PCR reactions were as follows: the initial denaturation at 95° C. for 5 minutes; the 1^{st} to 40^{th} cycles with one cycle including denaturation at 95° C. for 30 sec, annealing at 63° C. for 15 sec, and extension at 72° C. for 3 minutes; and then the final extension at 72° C. for 3 minutes and storage at 4° C.

The results are shown in FIG. 4C. The arrangement of the capture probes on the microarray in FIG. 4C is described in Table 4.

TABLE 4
Arrangement of capture probes on the microarray

| | Column 1 | Column 2 | Column 3 | Column 4 | Column 5 | Column 6 | Column 7 | Column 8 |
|---|---|---|---|---|---|---|---|---|
| Row 1 | + | − | MO1E02- | MO1E02- | MO1E02- | MO1E02- | MO1E03- | MO1E03- |
| | | | 01wp | 01mp | 02wp | 02mp | 01wp | 01mp |
| Row 2 | MO1E03- | MO1E03- | MO1E04- | MO1E04- | MO1E04- | MO1E04- | MO1E04- | MO1E04- |
| | 02wp | 02mp | 01wp | 01mp | 02wp | 02mp | 03wp | 03mp |
| Row 3 | MO1E04- | MO1E04- | MO1E07- | MO1E07- | MO1E07- | MO1E07- | MO1E07- | MO1E07- |
| | 04wp | 04mp | 01wp | 01mp | 02wp | 02mp | 03wp | 03mp |
| Row 4 | MO1E08- | MO1E08- | MO1E09- | MO1E09- | MO1E10- | MO1E10- | | |
| | 01wp | 01mp | 01wp | 01mp | 01wp | 01mp | | |

As illustrated in FIGS. 4A, 4B, and 4C, whereas the background fluorescent intensity of the microarray in which target nucleic acids were amplified by a conventional direct labeling PCR method was an average of 150 (see, FIG. 4C), those of the microarrays in which target nucleic acids were amplified by the detection methods of the present invention were respectively an average of 106 and 60. That is, the latter showed a remarkable reduction in the background fluorescent intensity (see, FIGS. 4A and 4B). In particular, the detection method comprising hybridizing the detection probes and the hybridization products obtained by hybridizing the PCR products with the capture probes, among the detection methods of the present invention, exhibited background fluorescent intensity as less as 40% of that of the conventional direct labeling method. Thus, the detection method of the present invention may provide reduced background fluorescent intensity and thus provide the effects of increasing the ratio of signal to noise.

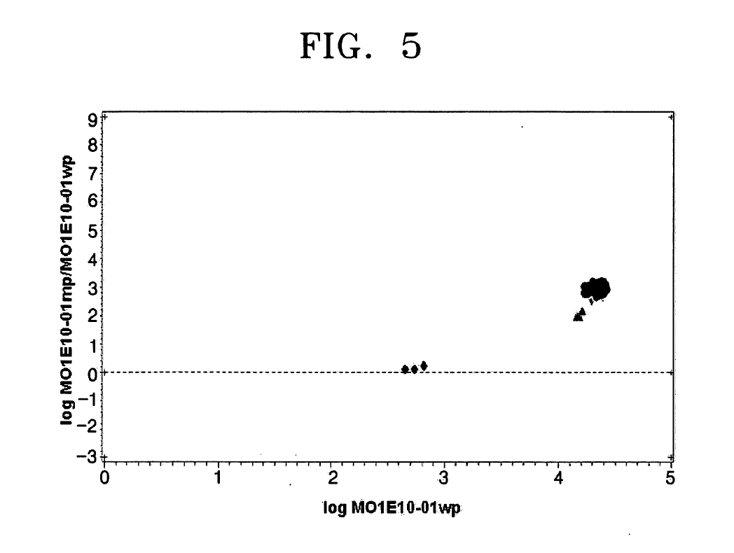

Fluorescent signals of the capture probe spots were analysed for detecting mutation of a specific gene, using the resulting image data. A graph was plotted and analysed, in which the x-axis represents a log of a fluorescent signal (wp) of the capture probe spots for detecting a normal type and the y-axis of y represents a log of the ratio (mp/wp) of a fluorescent signal (mp) of the capture probe spots for detecting a mutant type to a fluorescent signal (wp) of the capture probe spots for detecting a normal type. For example, the analytical results of fluorescent signals for spots of 1454V mutant of exon 10 and capture probe for the detection of its normal type, i.e., MO1E10-01mp and MO1E10-01wp, are shown in FIG. 5.

In FIG. 5, ● represents analytical data of fluorescent intensity of targets amplified according to the direct labeling method (4), ▴ represents those amplified according to the method (2), and ▾ represents those amplified according to the method (3). Also, ♦ represents an analytical data of fluorescent intensity of PCR control. As seen from FIG. 5, fluorescent intensity of targets amplified according to the method (2) or (3) was roughly similar to those according to the conventional direct labeling method (4). Specifically, fluorescent intensity of targets amplified according to the method (3) is almost identical to those according to the conventional method.

As described above, the method for detecting target nucleic acids according to the present invention may provide almost similar effects in terms of fluorescent intensity, compared with the conventional direct labeling method (see, FIG. 5). Also, the method of the present invention may provide excellent effects in terms of background signals, compared with the conventional direct labeling method.

According to the method for detecting target nucleic acids of the present invention, even a plurality of target nucleic acids amplified by a multiplex PCR method may be detected using the same detection probes. Also, with regard to the increase in cost, which is proportional to the number of type of target nucleic acids, the present method has a relatively low cost increase rate than the conventional method. Further, the method of the present invention may be performed in a simpler process since it does not need a separate labeling reaction.

The amount of commercially available fluorescent dyes used in the method of the present invention is just about 2% of that in the conventional direct labeling method, which is advantageous in view of economy. Also, the method of the present invention may prevent the adverse effects of delaying or stopping the PCR as occurred in the direct labeling method.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method for detecting a target nucleic acid, comprising:
amplifying a target nucleic acid from a nucleic acid sample using a set of primers comprising at least one primer having a tag sequence at its 5′ end and a target binding sequence at its 3′ end;
hybridizing the resulting amplified products with detection probes labeled with a label, the detection probes being capable of specifically binding to the tag sequence;
hybridizing the hybridization products with capture probes immobilized on a microarray, the capture probes being capable of specifically binding to the target nucleic acid and not to the tag sequence; and
determining the results of hybridization.

2. The method of claim 1, wherein the amplification is performed by a PCR or SDA method.

3. The method of claim 1, wherein the amplification is performed by a multiplex PCR method.

4. The method of claim 1, wherein the label is selected from the group consisting of a fluorescent label, a radioactive, a receptor, and a ligand.

5. The method of claim 1, wherein the results of hybridization are determined by measuring a signal generated from the label of the detection probes.

6. A method for detecting a target nucleic acid, comprising:
amplifying a target nucleic acid from a nucleic acid sample using a set of primers comprising at least one primer having a tag sequence at its 5′ end and a target binding sequence at its 3′ end;
hybridizing the resulting amplified products with capture probes immobilized on a microarray, the capture probes being capable of specifically binding to the target nucleic acid and not to the tag sequence;
hybridizing the hybridization products with detection probes labeled with a label, the detection probes being capable of specifically binding to the tag sequence; and
determining the results of hybridization.

7. The method of claim 6, wherein the amplification is performed by a PCR or SDA method.

8. The method of claim 6, wherein the amplification is performed by a multiplex PCR method.

9. The method of claim 6, wherein the label is selected from the group consisting of a fluorescent label, a radioactive, a receptor, and a ligand.

10. The method of claim 6, wherein the results of hybridization are determined by measuring a signal generated from the label of the detection probes.

11. A method for detecting a target nucleic acid, comprising:
amplifying a target nucleic acid from a nucleic acid sample using a set of primers comprising at least one primer having a tag sequence at its 5′ end and a target binding sequence at its 3′ end;
hybridizing the resulting amplified products with detection probes labeled with a label and capture probes immobilized on a microarray, the detection probes being capable of specifically binding to the tag sequence and the capture probes being capable of specifically binding to the target nucleic acid and not to the tag sequence; and
determining the results of hybridization.

12. The method of claim 11, wherein the amplification is performed by a PCR or SDA method.

13. The method of claim 11, wherein the amplification is performed by a multiplex PCR method.

14. The method of claim 11, wherein the label is selected from the group consisting of a fluorescent label, a radioactive, a receptor, and a ligand.

15. The method of claim 11, wherein the results of hybridization are determined by measuring a signal generated from the label of the detection probes.


SEQUENCE LISTING (46 sequences)

SEQ ID NO: 1 - DNA, 15 bp, Artificial Sequence
  Tag sequence 1
tgttctcttgtcttg

SEQ ID NO: 2 - DNA, 15 bp, Artificial Sequence
  Tag sequence 2
atcggtttgtttgtc

SEQ ID NO: 3 - DNA, 15 bp, Artificial Sequence
  detection probe 1 coupled with Cy3 dye at the 5′ terminal
caagacaagagaaca

SEQ ID NO: 4 - DNA, 15 bp, Artificial Sequence
  detection probe 2 coupled with Cy3 at 5′ terminal
gacaaacaaaccgat

SEQ ID NO: 5 - DNA, 41 bp, Artificial Sequence
  primer
agcggaggccaggttttgcaccggctcccaccccagaaggt

SEQ ID NO: 6 - DNA, 44 bp, Artificial Sequence
  primer
aggcccgccactctgcaaaaccatgagcccaagtgtgcccattt

SEQ ID NO: 7 - DNA, 46 bp, Artificial Sequence
  primer
agcggaggccaggttttgcacccgggatgaagagatgagagcactg

SEQ ID NO: 8 - DNA, 44 bp, Artificial Sequence
  primer
aggcccgccactctgcaaaaggggcctgccactgagtcataaag

SEQ ID NO: 9 - DNA, 43 bp, Artificial Sequence
  primer
agcggaggccaggttttgcaagacacccccaccccctactcca

SEQ ID NO: 10 - DNA, 44 bp, Artificial Sequence
  primer
aggcccgccactctgcaaaagctgcaaactgggccatgtgaaac

SEQ ID NO: 11 - DNA, 43 bp, Artificial Sequence
  primer
agcggaggccaggttttgcaccctgcaggtcctcctcccacag

SEQ ID NO: 12 - DNA, 43 bp, Artificial Sequence
  primer
aggcccgccactctgcaaaaactgcgcccggccatattgtctc

SEQ ID NO: 13 - DNA, 45 bp, Artificial Sequence
  primer
agcggaggccaggttttgcaatgctggcgtaccctggttgttgag

SEQ ID NO: 14 - DNA, 42 bp, Artificial Sequence
  primer
aggcccgccactctgcaaaacaaagcggcacagtggggaagc

SEQ ID NO: 15 - DNA, 42 bp, Artificial Sequence
  primer
agcggaggccaggttttgcaggcgtcccaaggcctgaggtct

SEQ ID NO: 16 - DNA, 45 bp, Artificial Sequence
  primer
aggcccgccactctgcaaaacaatcttgccctttattccctaccc

SEQ ID NO: 17 - DNA, 41 bp, Artificial Sequence
  primer
agcggaggccaggttttgcaggcagggtgggaggggagaac

SEQ ID NO: 18 - DNA, 42 bp, Artificial Sequence
  primer
aggcccgccactctgcaaaagcgtcagggtgcagtgggatgt

SEQ ID NO: 19 - DNA, 14 bp, Artificial Sequence
  capture probe for wild type
ctgtgacggctgca

SEQ ID NO: 20 - DNA, 14 bp, Artificial Sequence
  capture probe for mutant type
ctgtgccggctgca

SEQ ID NO: 21 - DNA, 21 bp, Artificial Sequence
  capture probe for wild type
tgcaagggcttcttccggagg

SEQ ID NO: 22 - DNA, 21 bp, Artificial Sequence
  capture probe for mutant type
tgcaagggcttcccggaggag

SEQ ID NO: 23 - DNA, 19 bp, Artificial Sequence
  capture probe for wild type
tcctcttgtctttgtccac

SEQ ID NO: 24 - DNA, 19 bp, Artificial Sequence
  capture probe for mutant type
cactggttcctcgtctttg

SEQ ID NO: 25 - DNA, 13 bp, Artificial Sequence
  capture probe for wild type
cgggctggcatga

SEQ ID NO: 26 - DNA, 13 bp, Artificial Sequence
  capture probe for mutant type
cgggctagcatga

SEQ ID NO: 27 - DNA, 13 bp, Artificial Sequence
  capture probe for wild type
ctggacggctgtg

SEQ ID NO: 28 - DNA, 13 bp, Artificial Sequence
  capture probe for mutant type
tctggatggctgt

SEQ ID NO: 29 - DNA, 13 bp, Artificial Sequence
  capture probe for wild type
atccggtcccgct

SEQ ID NO: 30 - DNA, 13 bp, Artificial Sequence
  capture probe for mutant type
atccagtcccgct

SEQ ID NO: 31 - DNA, 19 bp, Artificial Sequence
  capture probe for wild type
ggtacctgtcgggacagga

SEQ ID NO: 32 - DNA, 18 bp, Artificial Sequence
  capture probe for mutant type
gtacctgtcaggacagga

SEQ ID NO: 33 - DNA, 19 bp, Artificial Sequence
  capture probe for wild type
cggtacctgtcgggacagg

SEQ ID NO: 34 - DNA, 18 bp, Artificial Sequence
  capture probe for mutant type
ggtacctgttgggacagg

SEQ ID NO: 35 - DNA, 13 bp, Artificial Sequence
  capture probe for wild type
gacacccggctca

SEQ ID NO: 36 - DNA, 13 bp, Artificial Sequence
  capture probe for mutant type
tggacatccggct

SEQ ID NO: 37 - DNA, 14 bp, Artificial Sequence
  capture probe for wild type
ctcctggaagggca

SEQ ID NO: 38 - DNA, 14 bp, Artificial Sequence
  capture probe for mutant type
agctcctagaaggg

SEQ ID NO: 39 - DNA, 17 bp, Artificial Sequence
  capture probe for wild type
aggcatactcattgtca

SEQ ID NO: 40 - DNA, 17 bp, Artificial Sequence
  capture probe for mutant type
aggcatactgattgtca

SEQ ID NO: 41 - DNA, 13 bp, Artificial Sequence
  capture probe for wild type
ccaaagcggccac

SEQ ID NO: 42 - DNA, 13 bp, Artificial Sequence
  capture probe for mutant type
ccaaagtggccac

SEQ ID NO: 43 - DNA, 14 bp, Artificial Sequence
  capture probe for wild type
acgatgacgttggt

SEQ ID NO: 44 - DNA, 14 bp, Artificial Sequence
  capture probe for mutant type
acgatgatgttggt

SEQ ID NO: 45 - DNA, 13 bp, Artificial Sequence
  capture probe for wild type
tgggggatggcag

SEQ ID NO: 46 - DNA, 13 bp, Artificial Sequence
  capture probe for mutant type
gggggacggcaga